(19) United States
(12) Patent Application Publication
(10) Pub. No.: US 2002/0139372 A1
(43) Pub. Date: Oct. 3, 2002

(54) Tracheostomy tube with adjustable quick release and method therefor

(76) Inventor: Alan H. Shikani, Ruxton, MD (US)

Correspondence Address: LEONARD BLOOM & ASSOCIATES, LLC., 401 Washington Avenue, Suite 905, Towson, MD, 21204, US

(21) Appl. No.: 09/801,486

(22) Filed: Mar. 8, 2001

(51) Int. Cl. A61M 16/00; A62B 9/06

(52) U.S. Cl. 128/207.17; 128/207.15

(57) ABSTRACT

A tracheostomy tube has a cannula and a neckpiece with bands. The distal ends of the bands are rapidly and releasably connected to one another. At least one of the bands is adjustable in length. The bands and the neck piece may be formed integrally into a unitary member. The bands and neck piece may be formed from a viscoelastic polymer. A method of use is disclosed.

### FIELD OF THE INVENTION

The present invention relates to a tracheostomy tube and more particularly, to a tracheostomy tube with a means for quickly attaching around the neck of the patient and easily adjusting for the comfort of the patient. The tracheostomy tube has a neck plate and is formed from a soft material which has improved patient comfort and decreases pressure on the skin.

### DESCRIPTION OF PRIOR ART

Tracheostomy tubes are usually held around the neck of the patient with a relatively thin cotton lacing which has ends tied to slotted openings in the opposite ends of the neck plate. The widely used 4LPC tracheostomy tube marketed by Shiley Incorporated, Irvine, Calif., and the model 60A 160 Adult Tracheostomy Tube Cuffless marketed by Bivona Medical Technologies, Gary, Ind., both this system. The lacing irrates the neck of the patient as it is worn and is difficult to tie so it is not too tight or too loose. In addition, one end of the lacing must be threaded in the slotted opening and tied into a knot while the patient is wearing the tracheostomy tube. In this process, the cannula in the incision may be moved and the patient suffers discomfort if not actual pain.

The 8 DCT tracheostomy tube from Shiley, Inc. has a fabric covered foam band with a strip of hook-type fastener attached to each end. The strip of fastener is looped from the neck side of the neck plate through a slot and the hook fastener engages the fabric covering. This is performed while the tracheostomy tube is inserted in the patient and may cause discomfort or pain to the patient.

This problem has existed for many years and the applicant is aware of numerous concepts to improve the tracheostomy tube. These include the following:

| U.S. Pat. No. | Inventor(s) |
|---|---|
| 1,835,757 | Burchett |
| 2,039,142 | Brehm |
| 2,765,792 | Nichols |
| 3,236,236 | Hudson |
| 3,987,798 | McGinnis |
| 4,313,437 | Martin |
| 4,331,144 | Wapner |
| 5,010,884 | Van Derdoes et al |
| 5,368,023 | Wolf |

The above references disclose tracheostomy tubes with various means of connecting the band to the neck plate and around the neck of the patient. These include tie tapes, engagement hooks, clips, hook and loop fasteners and other means. The references also disclose padded tie tapes. However, despite the recognition of the problem, and the longstanding need, there is no readily available product on the market which overcomes the problems.

### BRIEF SUMMARY OF THE INVENTION

It is an object of the present invention to provide a tracheostomy tube which can be custom-fitted to the patient's neck and can be adjusted for the comfort of the patient.

It is a further object to provide a tracheostomy tube formed from a soft pliable material which molds itself to the anatomy of the patient's neck.

In accordance with the teachings of the present invention there is disclosed a tracheostomy tube adapted to be disposed on the neck of a patient. A cannula is inserted into the throat of the patient. A neck plate has a center opening therein and the cannula is received in said center opening. The neck plate has two bands formed integrally thereon, the bands extending in opposite directions outwardly from the neck plate. Each band has a respective end. The respective ends of the bands have means thereon for being rapidly releasably connected to one another such that the bands encircle the neck of the patient.

In further accordance with the teachings of the present invention, there is disclosed a tracheostomy tube adapted to be disposed on the neck of a patient. A cannula is inserted into the neck of the patient. A neck plate having a first end, an opposite second end and a center opening receives the cannula in the center opening. A first band and a second band are provided. Each band has a respective first end and a second end. The first end of the first band is connected to the first end of the neckpiece. The first end of the second band is connected to the second end of the neck piece. Means are provided for rapidly connecting and disconnecting the second end of the first band with the second end of the second band. The connected bands encircle the patient's neck. An adjustment means is attached to at least one of the bands such that the at least one of the bands may be shortened or lengthened for the comfort of the patient.

In yet further accordance with the teachings of the present invention, there is disclosed a tracheostomy tube adapted to be disposed on the neck of a patient. A cannula is inserted into the throat of the patient. A neck plate is provided having a first end, an opposite second end and a center opening. The cannula is received in the center opening. A band is provided between the first end of the neck plate and the second end of the neck plate. The band has an adjustable length which may be shortened or lengthened to encircle the neck of the patient for the comfort of the patient. The band is formed from a viscoelastic polymer which molds itself to the anatomy of the patient's neck.

In still further accordance with the teachings of the present invention, there is disclosed a tracheostomy tube adapted to be disposed on the neck of a patient. A cannula is inserted into the throat of the patient. A neck plate is provided having a center opening therein. The cannula is received in said center opening. The neck plate is formed from a viscoelastomeric polymer.

In another aspect, there is disclosed a method of attaching a tracheostomy tube on the neck of a patient. A tracheostomy tube is provided having a cannula, a neck piece in which the cannula is received. A first band and a second band are attached to the neck piece at opposite ends of the neck piece. A means is provided for releasably connecting the two bands. At least one of the bands has an adjustment means. The cannula is inserted in an incision in the throat of the patient with the neck piece disposed against the throat of the patient. The bands are disposed around the neck of the patient and the bands are connected to one another with the connecting means. The at least one band is adjusted to comfortably fit the bands and the neck piece to the patient with minimal movement of the cannula within the incision.

These and other objects of the present invention will become apparent from a reading of the following specification taken in conjunction with the enclosed drawings.

### BRIEF DESCRIPTION OF THE DRAWINGS

FIG. 1 is a perspective view of a tracheostomy tube of the prior art.

FIGS. 2-4 is a sequence of perspective views showing the tying of the lacing of the prior art to the neck plate.

FIG. 2 shows the one end of the lacing tied to one side of the neck plate, the lacing encircling the neck and the other end of the lacing received in a slot in the other end of the neck plate.

FIG. 3 shows the other end of the lacing tied in a knot to the other end of the neck plate while the tracheostomy tube is on the patient's neck.

FIG. 4 shows the lacing tied to both ends of the neck plate.

FIG. 5 is a perspective view of the present invention showing a single means for both connecting and adjusting the length of the bands.

FIG. 6 is a perspective view of the present invention showing anther embodiment of a single means for both connecting and adjusting the length of the bands.

FIGS. 7-9 is a sequence of perspective views of the present invention showing the bands of the embodiment of FIG. 6 being connected and adjusted.

FIG. 7 shows the end of the one band threaded through the frame of the connector mounted on the other band.

FIG. 8 shows the end of the one band being back threaded through the frame of the connector.

FIG. 9 shows the end of the one band pulled to adjust the length of the one band.

FIG. 10 is a perspective view of the present invention showing an adjustment means and a quick connect/disconnect means.

FIG. 11 is a perspective view of a patient lying down with the quick connect/disconnect of the embodiment of FIG. 10 in place.

FIG. 12 is a perspective view of the present invention of FIG. 10 showing the bands formed of a viscoelastic polymer.

FIG. 13 is an enlarged cross-sectional view taken across the lines 13-13 of FIG. 12.

FIG. 14 is a perspective view of FIG. 12 showing another embodiment of the means for adjusting the length of the bands.

FIG. 15 is a perspective view of the present invention wherein the bands are integral with the neck plate and a connector/length adjuster is on the ends of the bands.

### DESCRIPTION OF THE PREFERRED EMBODIMENTS

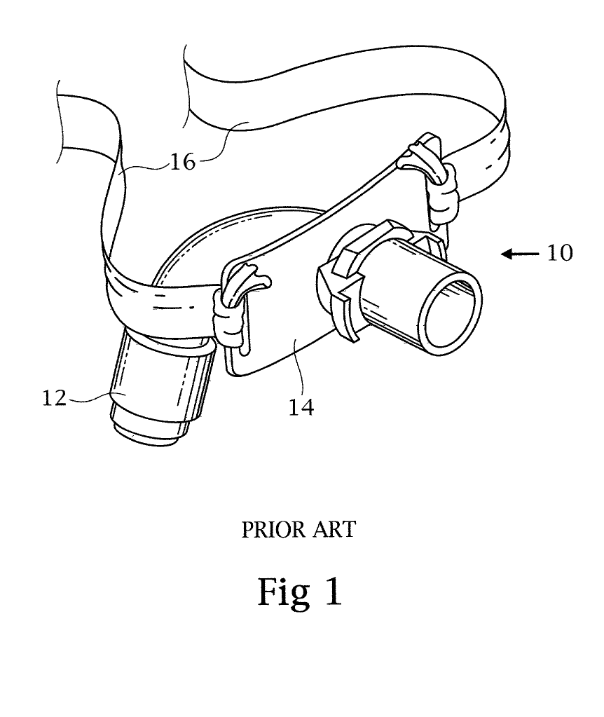

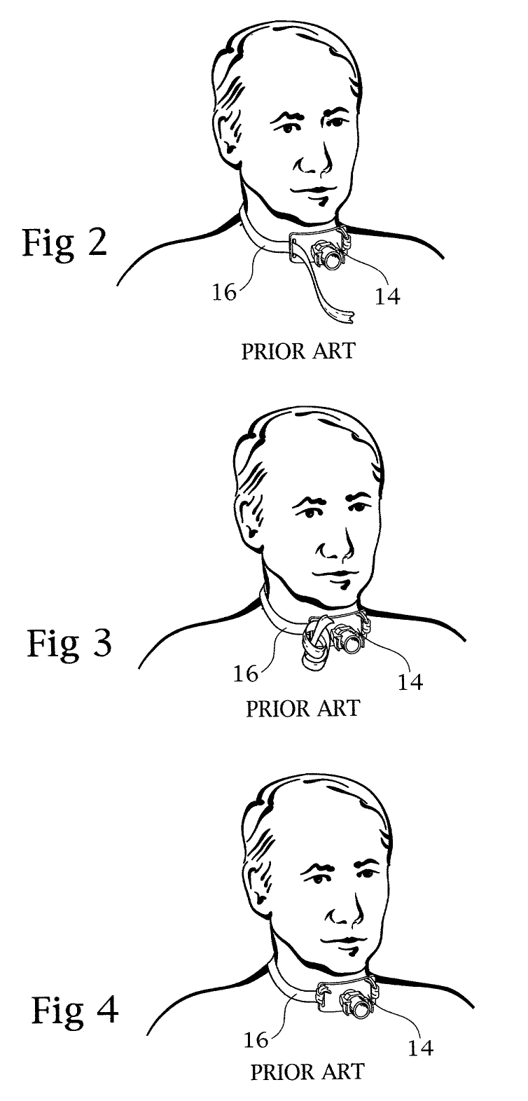

Referring now to FIGS. 1-4, a widely used tracheostomy tube of the prior art has a cannula 12 received in a center opening in a neck plate 14. At opposite ends of the neck plate 12, there are slotted openings. A first end of a lacing 16 is tied in one of the slotted openings and, when the tracheostomy tube is worn by the patient, the second end of the lacing 16 is placed around the neck of the patient and is inserted through the other slotted opening. The second end of the lacing 16 is tied to the neck plate. During the process, the tracheostomy tube is moved in several directions in the incision in the patient's throat with much discomfort and possibly pain. A comfortable fit around the patient's neck is difficult to obtain. The lacing may be too tight and irritate the skin or may be too loose and permit movement of the cannula.

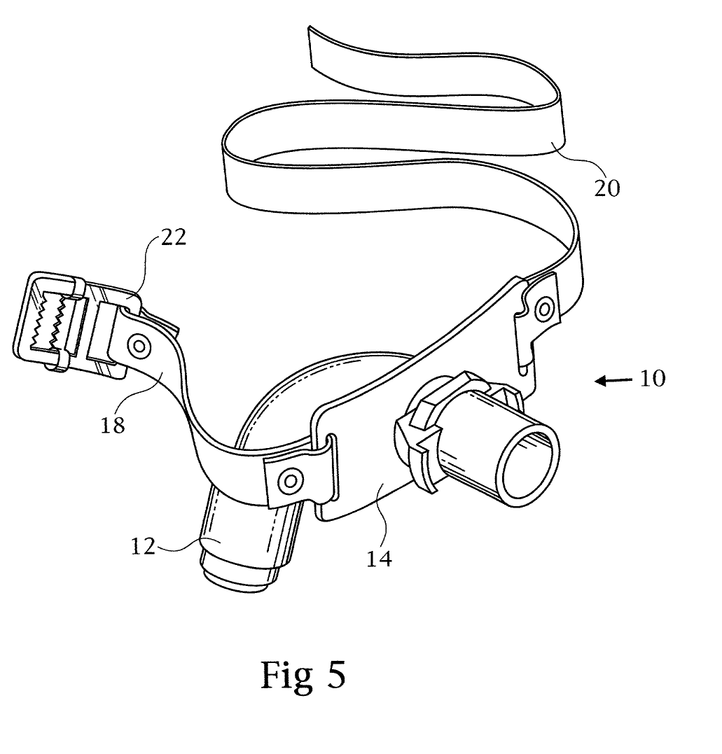

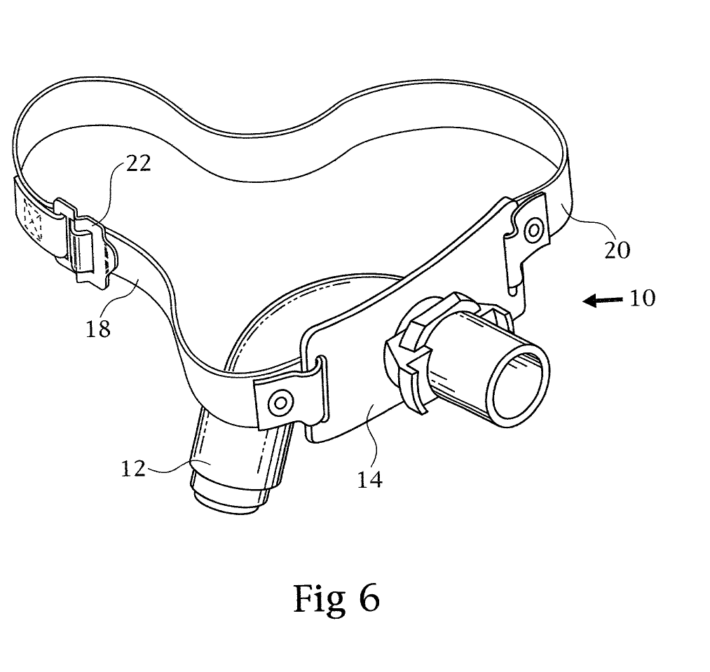

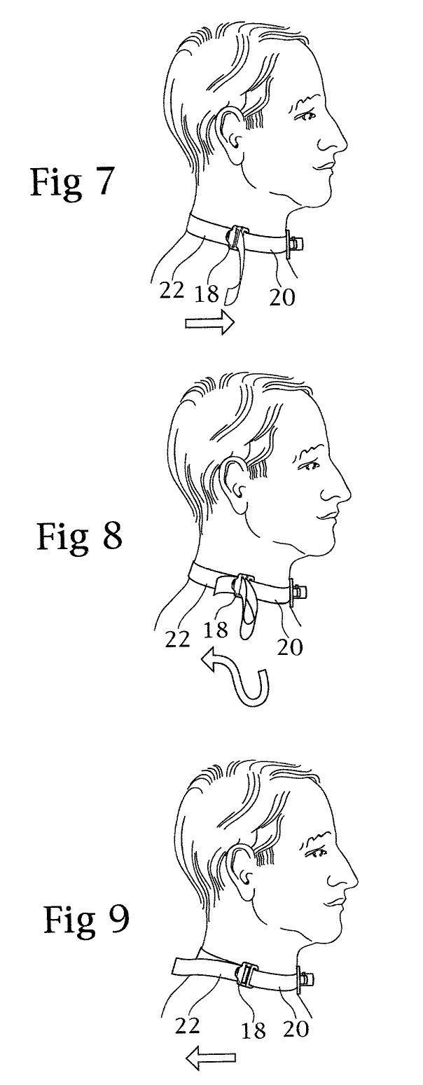

As shown in FIGS. 5 and 6, the present invention has a first band 18 connected to the neck plate 14 and a second band 20 connected to the opposite end of the neck plate 14. The end of the first band 18, distal from the neck plate 14, has a single means 22 for adjusting and connecting the bands 18, 20 attached thereto. FIGS. 6-9 show the tracheostomy tube of the embodiment of FIG. 6 of the present invention being attached to a patient. The cannula is received in the incision in the patient's throat and the bands 18, 20 are brought around the neck to meet on the side of the patient's neck. The distal end of the second band 20 is threaded through the adjusting means 20 (see arrow FIG. 7) turned back on itself and re-threaded through the adjusting means (see arrow FIG. 8) to retain the tracheostomy tube 10 on the patient's neck and to connect the bands 18, 20. The adjusting means 22 shown in FIGS. 6-9 also serves as a connecting means in that by drawing on the distal end of the second band 20 (see arrow FIG. 9) an increased length of the second band 20 is drawn through the connector 22 and the bands are tightened around the patient's neck. In this manner the band is shortened until the patient is comfortable and the tracheostomy tube is retained on the patient. The adjusting means 24 is not limited to the one shown in FIGS. 5-9 but may be any adjusting means 24 known to persons skilled in the art which provides a simple and easily manipulated means to lengthen and shorten at least one of the bands (18, 20). The at least one band need not be formed of two segments but may be an uninterrupted band.

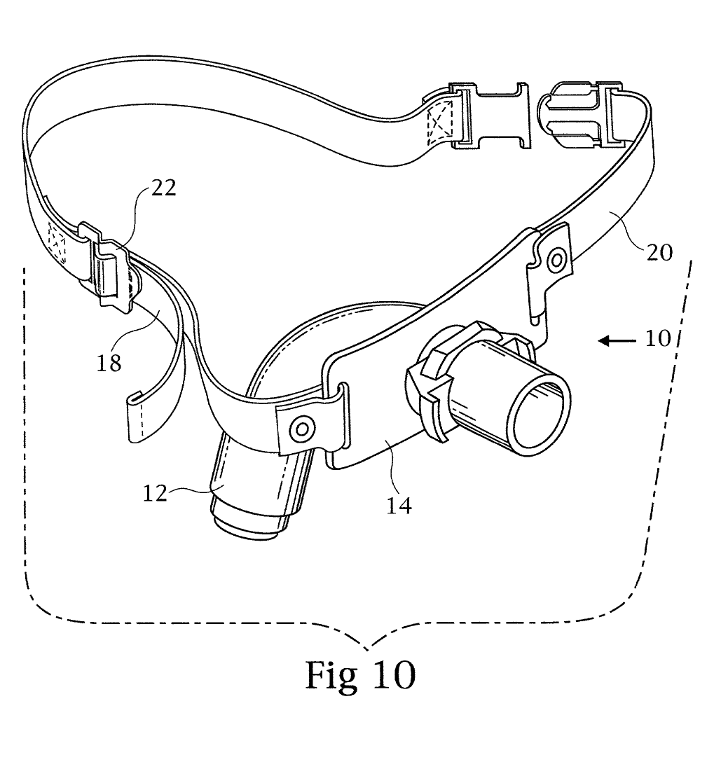

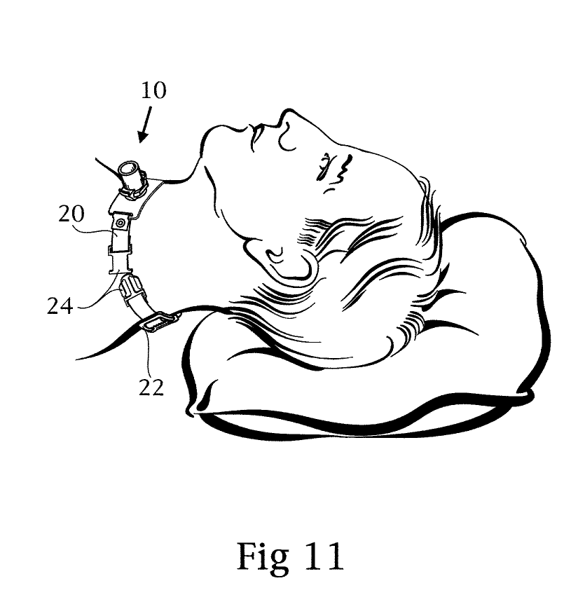

The adjusting means 22 and the connecting means 24 may be separated as shown in FIG. 10. The first band 18, second band 20 and adjusting means are the same as shown in FIGS. 6-9. However, as noted above, the adjusting means 24 is not so limited. A typical connecting means 24 has two releasable interlocking members which are quickly and easily connected and disconnected. Here, also, connecting means 24 known to persons skilled in the art may be used. It is preferred that one member of the connecting means 24 be attached to the distal end of the first band and the other member of the connecting means 24 be attached to the distal end of the second band 20. This does not preclude the connecting means 24 being disposed intermediately in one of the bands 18, 20. FIG. 11 shows the tracheostomy tube 10 of FIG. 10 mounted on a supine patient with the connecting means 24 in a disconnected position. Pushing the two members together to connect the members is accomplished very rapidly without moving the cannula 12 and with no discomfort to the patient.

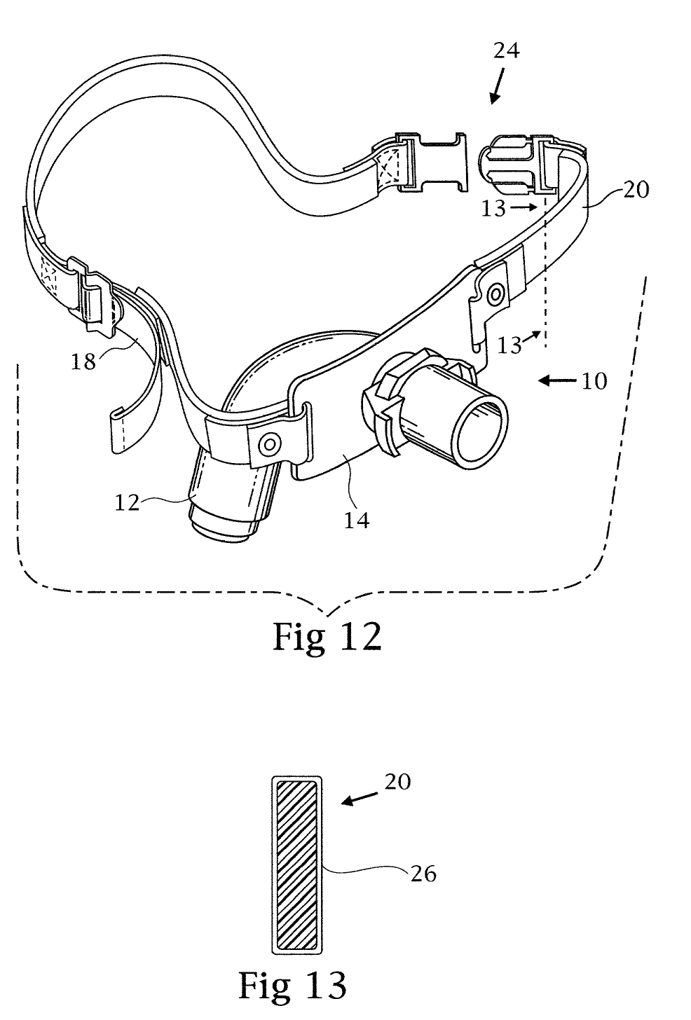

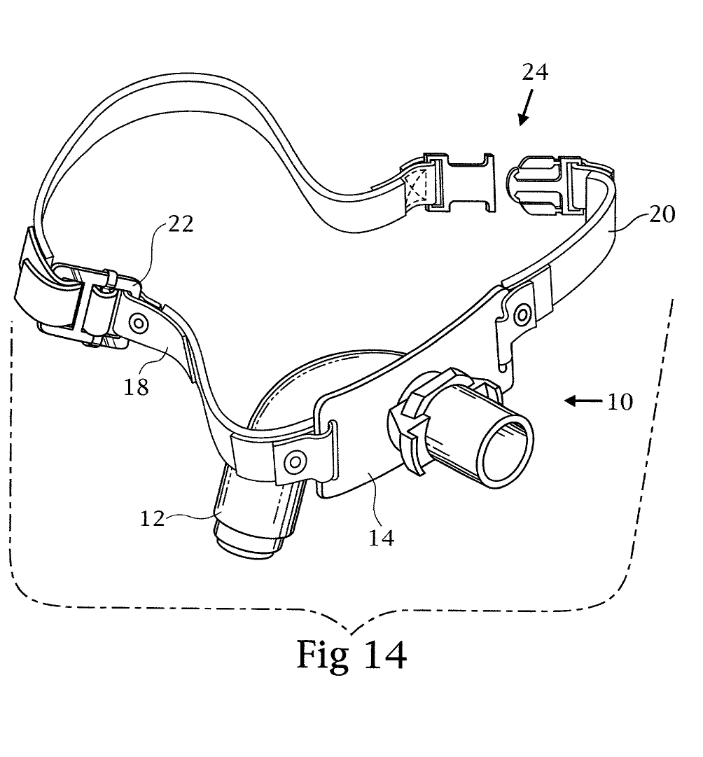

As shown in FIGS. 12-14, the present invention may have bands 18, 20 formed from a viscoelastic polymer which may be gel-like. The polymer is contained within a pliable encasing film 26. The nature of the polymer is to assume the configuration of the surface which it contacts. Thus, the bands mold themselves to the anatomy of the patient's neck and are very comfortable. A product sold as AKTON® by Action Products, Inc., Hagerstown, Md. 21740 may be used to form the bands.

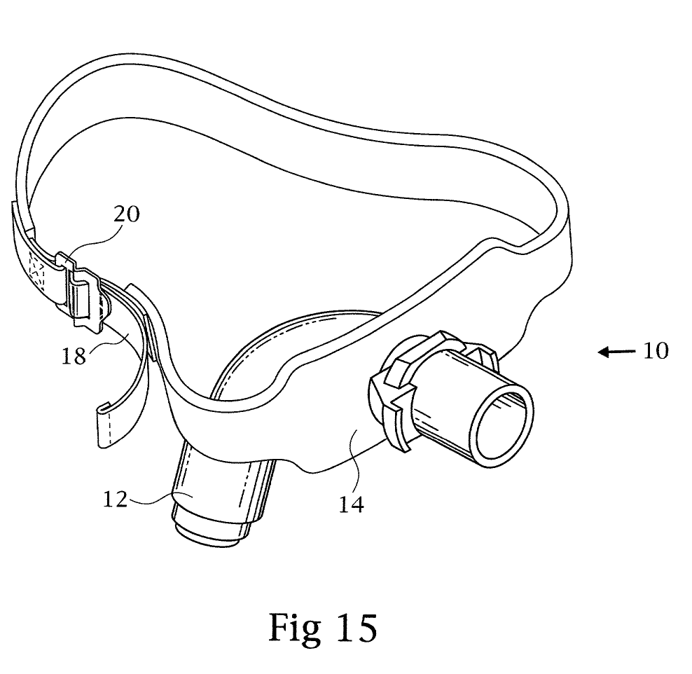

The neck plate 14 may be formed integrally with the first band 18 and the second band 20 to form a unitary member as shown in FIG. 15. Although FIG. 15 shows the unitary member to be formed from the viscoelastic polymer, it may be formed from other materials to which the connecting means 24 and/or the adjusting means 22 may be attached.

The tracheostomy tube 10 of the present invention is used by inserting the cannula 12 into an incision in the patient's throat with the neck plate 14 disposed against the patient's throat surrounding the cannula. When the neck piece is formed from the viscoelastic polymer, the irritation of the patient's throat around the incision is greatly reduced. The bands 18, 20 are placed on opposite sides of the patient's neck and the bands are connected to one another with the quick connect/disconnect connecting means 24. At least one of the bands is adjusted for length with the adjusting means 22 to secure the tracheostomy tube securely on the patient's neck without being too tight to be uncomfortable but still sufficiently tight to prevent slippage when the patient moves. The connecting means 24 and the adjusting means 22 are used with minimal movement of the cannula 12 in the incision and with no discomfort to the patient.

Obviously, many modifications may be made without departing from the basic spirit of the present invention. Accordingly, it will be appreciated by those skilled in the art that within the scope of the appended claims, the invention may be practiced other than has been specifically described herein.

What is claimed is:

1. A tracheostomy tube adapted to be disposed on the neck of a patient comprising:
a cannula inserted into the throat of the patient,
a neck plate having a center opening therein, the cannula being received in said center opening, the neck plate having two bands formed integrally thereon, the bands extending in opposite directions outwardly from the neck plate,
each band having a respective end, the respective ends of the bands having means thereon for being rapidly releasably connected to one another such that the bands encircle the neck of the patient.

2. The tracheostomy tube of claim 1, wherein the neck plate and the bands are formed from a viscoelastic polymer which molds itself to the anatomy of the patient's neck.

3. The tracheostomy tube of claim 1, further comprising an adjustment means attached to at least one of the bands such that the at least one of the bands may be shortened or lengthened for the comfort of the patient.

4. A tracheostomy tube adapted to be disposed on the neck of a patient, comprising:
a cannula inserted into the neck of the patient,
a neck plate having a first end, an opposite second end, and a center opening, the cannula being received in the center opening,
a first band and a second band, each band having a respective first end and a second end, the first end of the first band being connected to the first end of the neck piece, the first end of the second band being connected to the second end of the neck piece,
means for rapidly connecting and disconnecting the second end of the first band with the second end of the second band, the connected bands encircling the patient's neck, and
an adjustment means attached to at least one of the bands such that the at least one of the bands may be shortened or lengthened for the comfort of the patient.

5. The tracheostomy tube of claim 4, wherein the bands are formed from a viscoelastic polymer which molds itself to the anatomy of the patient's neck.

6. The tracheostomy tube of claim 5, wherein the neck plate is integral with the two bands forming a unitary member.

7. A tracheostomy tube adapted to be disposed on the neck of a patient comprising:
a cannula inserted into the throat of the patient,
a neck plate having a first end, an opposite second end and a center opening, the cannula being received in the center opening,
a band between the first end of the neck plate and the second end of the neck plate, the band to encircle the neck of the patient, having an adjustable length which may be shortened or lengthened for the comfort of the patient, and
the band being formed from a viscoelastic polymer which molds itself to the anatomy of the patient's neck.

8. The tracheostomy tube of claim 7, wherein the neck plate is integral with the band forming a unitary member.

9. A tracheostomy tube adapted to be disposed on the neck of a patient comprising:
a cannula inserted into the throat of the patient,
a neck plate having a center opening therein, the cannula being received in said center opening, the neck plate being formed from a viscoelastic polymer.

10. The tracheostomy tube of claim 9, further comprising the neck plate having two bands formed thereon, the bands being integral with the neck plate and extending in opposite directions outwardly from the neck plate, the bands encircling the neck of the patient, the bands being formed from a viscoelastic polymer.

11. The tracheostomy tube of claim 10, further comprising an adjustment means attached to at least one of the bands such that the at least one of the bands may be shortened or lengthened for the comfort of the patient.

12. A tracheostomy tube adapted to be disposed on the neck of a patient comprising:
a cannula inserted into the throat of the patient,
a neck plate having a center opening therein, the cannula being received in said center opening, the neck plate having two bands formed integrally thereon, the bands extending in opposite directions outwardly from the neck plate, the neck plate and the bands being formed from a viscoelastic polymer which molds itself to the anatomy of the patient's neck,
each band having a respective end, the respective ends of the bands having means thereon for being rapidly releasably connected to one another such that the bands encircle the neck of the patient, and
an adjustment means attached to at least one of the bands such that the at least one of the bands may be shortened or lengthened for the comfort of the patient.

13. A method of attaching a tracheostomy tube on the neck of a patient comprising the steps of:
providing a tracheostomy tube having a cannula, a neck piece in which the cannula is received, a first band and a second band attached to the neck piece at opposite ends of the neck piece, a means for releasably connecting the two bands and at least one of the bands having an adjustment means,
inserting the cannula in an incision in the throat of the patient with the neck piece disposed against the throat of the patient,
disposing the bands around the neck of the patient and connecting the bands to one another with the connecting means,
adjusting the at least one band to comfortably fit the bands and the neck piece to the patient with minimal movement of the cannula within the incision.

14. In a tracheostomy tube device, the improvement comprising a quick connect/quick disconnect strap provided with adjustability for easily fitting the device to a patient, and the strap being made from a relatively soft material for patient comfort.

15. The improvement of claim 14, wherein the strap is made from a viscoelastic polymer.

16. The improvement of claim 15, further including a neck plate for the device, the neck plate being made from a viscoelastic polymer and being formed integrally with the strap.

17. The improvement of claim 16, wherein the strap has two portions, each of which is formed integrally with a respective end of the neck plate.
